(19) United States
(12) Patent Application Publication
(10) Pub. No.: US 2014/0343452 A1
(43) Pub. Date: Nov. 20, 2014

(54) Pseudobezoar-Based Intraluminal Gastrointestinal Cleansing and Biospy

(71) Applicant: EatLittle Inc., Calgary (CA)

(72) Inventors: Martin P. Mintchev, Calgary (CA); Orly Yadid-Pecht, Calgary (CA)

(73) Assignee: EatLittle Inc., Calgary (CA)

(21) Appl. No.: 14/367,289

(22) PCT Filed: Dec. 13, 2012
(86) PCT No.: PCT/CA2012/050896

§ 371 Date: Jun. 20, 2014

Related U.S. Application Data
(60) Provisional application No. 61/630,816, filed on Dec. 21, 2011.

(51) Int. Cl. A61B 10/04; A61B 10/00; A61M 29/02

(52) U.S. Cl. CPC A61B 10/04; A61M 29/02; A61B 10/0045; A61B 2010/0061
USPC 600/562; 606/192

(57) ABSTRACT

The present disclosure describes an orally administrable implement comprising expandable material designed specifically to swell in a targeted gastrointestinal (Gl) organ of a mammal, including human to form a pseudobezoar, so that upon swelling it touches the walls of the targeted organ. In one design, the swollen pseudobezoar provides sponge-like cleansing of the targeted Gl organ to prepare the said organ for further examination. In another design the expandable material scrapes the walls of the targeted Gl organ by creating a sufficient friction tension between its permeable wails and the mucosa of the Gl organ walls to collect enough adequate tissue samples, occult blood, or both, for the purpose of tissue sample analysis, DNA analysis, occult blood analysis, or a combination thereof. Safety mechanisms for the controlled disintegration of the pseudobezoars in cases of possible obstruction are also described.

### 1. FIELD OF THE INVENTION

The present invention relates to the field of ingestible medical devices, and, more specifically, to an orally administrable implement comprising expandable structure designed specifically to swell in a targeted gastrointestinal (GI) organ of a mammal, including human to form a pseudobezoar, so that upon swelling it touches the walls of the targeted organ. In one design, the swollen pseudobezoar provides sponge-like cleansing of the targeted GI organ to prepare the said organ for further examination, followed, for example, by the administration of a second pseudobezoar, which upon swelling in the targeted organ creates a sufficient friction tension between its permeable walls and the mucosa of the GI organ walls to collect enough adequate tissue samples, occult blood, or both, for the purpose of tissue sample analysis, DNA analysis, occult blood analysis, or a combination thereof. Both swollen pseudobezoars are expelled from the body in a natural way and at least one of them contains valuable tissue samples, occult blood, or both. Upon exiting the body, both pseudobezoars are collected and sent for standard tissue sample, occult blood, DNA analysis, or a combination thereof. In another design, a single pseudobezoar can be administered for cleansing the targeted GI organ only. In yet another design, a single pseudobezoar can be administered for collecting tissue samples or occult blood from the targeted GI organ only. A safety mechanism for the controlled disintegration of the pseudobezoars in cases of possible obstruction is also described.

### 2. BACKGROUND OF THE INVENTION

In the present disclosure we will discuss the colon as the targeted GI organ of interest. However, the described methodology is applicable to all other organs in the GI tract, e.g. the throat, the esophagus, the stomach, the duodenum and the small intestine. Organ-targeting shell cover is known in the art.

2.1. Colon and Colon Cancer

Colon cancer is the most common gastrointestinal (GI) malignancy and the second leading cause of cancer deaths in the United States (Jemal et al., CA Cancer J Clin, 2008; 58(2):71-96). Of the many pre-neoplastic and neoplastic conditions in humans, nowhere is the ability to prevent disease as profound as it is in colon cancer (Yang et al., Gastroenterology 2010, 138(6):2027-2028). Strategies for prevention have evolved over the past 15 years, now including the use of fecal occult blood test, fecal immunology tests, fecal DNA tests, colonoscopy, video capsule endoscopy (VCE), and computed tomographic (CT) colonography, also known as Virtual Colonoscopy (Weizman a. Nguyen, Minerva Gastroenterologica e Dietologica 2010; 56(2):181-188).

2.2. Tests for Colon Cancer

2.2.1. Fecal Occult Blood Tests.

Although improved fecal occult blood tests have been utilized (see e.g. U.S. Pat. No. 4,615,982), the overall sensitivity of this approach is not impressive. In a 2005 study Morikawa et al (Gastroenterology 2005; 129:422-428) concluded that the sensitivity of 1-time immunochemical FOBT for detecting advanced neoplasia and invasive cancer was 27.1% and 65.8%, respectively. In addition, the sensitivity for invasive cancer according to Dukes' stage showed 50.0% for Dukes' stage A, 70.0% for Dukes' stage B, and 78.3% for Dukes' stages C or D (Zinkin L D, Diseases of the Colon & Rectum 1983, 26(1):37-43). The sensitivity for detecting advanced neoplasia in the proximal colon was significantly lower than that detected in the distal colon (16.3% vs 30.7%, P<0.00007). Recent US patent applications have tried to improve this type of testing (20100323367, 20090291447, 20060216714, 20050118657).

2.2.2. Fecal Immunology Tests.

This testing method can be considered a refinement, extension and an additive improvement of the traditional fecal occult blood testing (see e.g. U.S. Pat. No. 4,789,629). It has been reported that when a routine fecal occult blood test (e.g. a sensitive guaiac test) is combined with an immunological test for human haemoglobin the sensitivity improves to 97% (only 3% false negative results) in patients and no false positives in controls (Turunen et al. Br J Cancer 1984; 49(2): 141-148). Another far more comprehensive study (Allison et al., N Engl J Med 1996; 334(1)155-160) found that the sensitivity of the combined test was the highest among all occult blood tests (in the range of 80%), and its specificity for detecting cancer was above 97%.

The problem of all fecal occult blood tests, however, is that they aim at discovering blood in the feces resulting from existing bleeding colorectal lesions, while adenomatous polyps in asymptomatic average-risk adults remain undetected. Therefore, by the time findings are obtained with the fecal tests, it is usually too late (Levin et al., Gastroenterology, 2008, 134(5): 1570-1595; Levin et al., Cancer 2002, 95(8): 1618-1628). Nevertheless, the use of either annual or biennial fecal occult-blood testing significantly reduces the incidence of colorectal cancer (Mandel et al, N Engl J Med 2000; 343:1603-1607). An immunological assay and kit for colon cancer screening based on glycoprotein analysis has, therefore, been disclosed (US Patent Application No. 20020009760). Glucoproteins are extracted from individual samples such that immunogenicity is maintained. The purified fecal glycoproteins are reacted with antibodies to Colon and Ovarian Tumor Antigen (COTA).

2.2.3. Fecal DNA Tests.

Oncogene mutations that characterize colorectal neoplasia are detectable in exfoliated epithelial cells in the stool. Whereas neoplastic bleeding is intermittent making the detection of occult fecal blood more or less random, epithelial shedding is continual, potentially making fecal DNA testing more sensitive. Early XXI century reports indicated that a fecal DNA test had a sensitivity of 91 percent for the detection of colorectal cancer and 82 percent for the identification of adenomas (Woolf, N Engl J Med 2004; 351:2755-2758). However, one other report indicated that fecal DNA testing did not improve dramatically the preventive early detection of colonic cancer compared to occult fecal blood testing (Song et al., Gastroenterology 2004; 126(5), pp. 1270-1279). A list of recent US patent applications claiming various techniques to improve and perfect this technique is provided below:
1. 20100291124 Extracellular serine protease
2. 20100173300 NONCONTACT STIRRING METHOD, NONCONTACT STIRRING APPARATUS, METHOD AND APPARATUS FOR REACTING NUCLEIC ACID HYBRIDIZATION USING THE APPARATUS, METHOD FOR DETECTING NUCLEIC ACID IN SAMPLE, APPARATUS FOR DETECTING NUCLEIC ACID, METHOD FOR DETECTING ANTIBODY IN SAMPLE, APPARATUS FOR DETECTING ANTIBODY
3. 20100143943 DETECTION OF DYSPLASTIC OR NEOPLASTIC CELLS USING ANTI-MCM2 ANTIBODIES
4. 20100137618 METHOD FOR SCREENING FOR COMPOUNDS SELECTIVELY INTERACTING WITH RADS
5. 20100137236 Compositions Comprising a GPR109 Ligand For Treating Disorders of the Digestive Tract and/or Cancer
6. 20100119514 Antibodies Against Cancer
7. 20100092981 METHODS OF DETECTING HYPERMETHYLATION
8. 20100069255 METHOD FOR IDENTIFYING THERAPEUTICAL TARGETS IN SECONDARY TUMORS, THE USE OF THEREOF AND MEANS FOR IDENTIFYING, LABELLING AND TARGETING SECONDARY TUMORS
9. 20100068720 Method and kit for detection of early cancer or pre-cancer using blood and body fluids
10. 20090269757 DIAGNOSIS KITS AND METHOD FOR DETECTING CANCER USING POLYMORPHIC MINISATELLITE
11. 20090226936 Tetrahydrofolate Synthetase Gene
12. 20090123470 Antibodies Against Cancer
13. 20090098542 Gene Methylation in Colon Cancer Diagnosis
14. 20090074800 Cancer antigen and use thereof
15. 20090030088 Therapeutic benefits of gossypol, 6-methoxy gossypol, and 6,6′-dimothxy gossypol
16. 20080152633 Flavivirus Replicon Constructs for Tumor Therapy
17. 20080138329 Inhibitors of Dna Methylation in Tumor Cells
18. 20080085867 Early detection and prognosis of colon cancers
19. 20070275421 Detection of dysplastic or neoplastic cells using anti-MCM2 antibodies
20. 20070212369 Colon Specific Genes and Proteins
21. 20070184438 Methods and nucleic acids for the analysis of colorectal cell proliferative disorders
22. 20070178108 Colon Specific Gene and Protein and Cancer
23. 20070141582 Method and kit for detection of early cancer or pre-cancer using blood and body fluids
24. 20070037159 Tetrahydrofolate synthetase gene
25. 20060251666 Cancer antigens and utilization thereof
26. 20060246433 Method and nucleic acids for the analysis of a colon cell proliferative disorder
27. 20060240414 Genetically engineered glutaminase and its use in antiviral and anticancer therapy
28. 20060234342 Human DNA topoisomerase 1 alpha
29. 20060216713 Method for the identification of colorectal tumors
30. 20060205054 Extracellular serine protease
31. 20060099580 Methyl-cpg binding domain protein 2 homologs
32. 20060019277 Digital amplification for detection of mismatch repair deficient tumor cells
33. 20050287123 DNA vaccines encoding CEA and a CD40 ligand and methods of use thereof
34. 20050064410 Method and nucleic acids for the analysis of colon cancer
35. 20040265833 Methods and nucleic acids for the analysis of colorectal cell proliferative disorders
36. 20040254101 Colon specific gene and protein and cancer
37. 20040176576 Antibodies against cancer
38. 20040132976 Colon specific genes and proteins
39. 20040006054 Cytotoxic N-unsubstituted indoles and cyclopent(b)indoles and method of making and using same
40. 20030199010 Extracellular serine protease
41. 20030180765 Digital amplification for detection of mismatch repair deficient tumor cells
42. 20030176377 DNA vaccines encoding CEA and a CD40 ligand and methods of use thereof
43. 20030162195 Prediction of cancer by detection of ATM mutations
44. 20030158098 Colon specific gene and protein
45. 20030143646 Detection of dysplastic or neoplastic cells using anti-MCM2 antibodies
46. 20030099686 Theobromine with an anti-carcinogenic activity
47. 20020132233 Development of immuno-PCR for serological diagnosis of gastric carcinoma
48. 20020086314 Colon specific genes and proteins
49. 20020064862 Genetically engineered glutaminase and its use in antiviral and anticancer therapy
50. 20020061527 Human DNA topoisomerase 1 alpha
51. 20020037581 Extracellular serine protease

2.2.4. Colonoscopy

Traditional colonoscopy has been considered a safe, reliable, real-time and quick method for assessing colonic abnormalities in. Moreover, it offers the ability to remove polyps during the procedure. Although classical colonoscopy can be considered safe, reliable, real-time and quick, recent population-based studies have demonstrated that the rate of protection against colorectal cancer that it offers was only 30 to 50% (Müller a. Sonnenberg, Arch Intern Med. 1995; 155(16):1741-1748). In addition, colonoscopy is an invasive procedure, performed in a hospital setting, requires extensive and expensive logistic preparations, carries substantial risks of harming patients (2-4/1000), is heavily operator-dependent, and requires post-procedural recovery (Weinberg, Annals of Internal Medicine, 2011, 154(1):68-69, 2011; Minoli et al., Endoscopy, 1999, 31(7):522-527, 1999). Recent US patent applications tried to improve various aspects of this technique, and are listed below:
1. 20110251454 Colonoscopy Tracking and Evaluation System
2. 20110128352 FAST 3D-2D IMAGE REGISTRATION METHOD WITH APPLICATION TO CONTINUOUSLY GUIDED ENDOSCOPY
3. 20110065991 HANDGRIP FOR ASSESSMENT OF COLONOSCOPE MANIPULATION
4. 20110065989 SYSTEM FOR ASSESSMENT OF COLONOSCOPE MANIPULATION
5. 20100304410 METHOD OF ASSESSING COLORECTAL CANCER STATUS IN AN INDIVIDUAL
6. 20100280318 Colonoscope Guide and Method of Use for Improved Colonoscopy
7. 20100198011 SELF-PROPELLABLE APPARATUS AND METHOD
8. 20100125169 DE-LOOPING TOOL FOR AN ENDOSCOPE
9. 20090306476 AUTOMATED CONTROL OF IRRIGATION AND ASPIRATION IN A SINGLE-USE ENDOSCOPE
10. 20090287056 Device to facilitate suctioning of fluid during gastrointestinal endoscopy
11. 20080045790 Self-propellable endoscopic apparatus and method
12. 20080027281 Colonoscope guide and method of use for improved colonoscopy
13 20070013710 Fast 3D-2D image registration method with application to continuously guided endoscopy
14. 20060270901 Endoscope propulsion system and method
15. 20060089533 Self-propellable endoscopic apparatus and method
16. 20060069306 Automated control of irrigation and aspiration in a single-use endoscope
17. 20050222494 Dual-scope colonoscopy system with separate secondary colonoscope tool
18. 20050119528 Colonoscope apparatus and method
19. 20040260150 Automated self-propelling endoscope
20. 20040204702 Propulsion mechanism for endoscopic systems
21. 20040097789 Colonoscope apparatus and method

2.2.5. Video Capsule Endoscopy.

Orally administered capsule endoscope (CE) is a simple, safe, non-invasive, and non-sedation requiring procedure. VCE is well accepted and tolerated by the patients and allows complete exploration of the small bowel. Usually, it takes 24 to 48 hours for a CE to pass through the entire GI tract as a result of its passive movement from mouth to anus [10]. In view of the fact that the movement of these capsules is controlled by spontaneous gut peristalsis, the application of VCE is currently limited to small-lumen organs [11]. In larger-lumen organs, such as the stomach or the colon, the capsules tend to tumble, which leads to incorrect recognition of a given organ segment by the capsule imaging system, thus rendering the images unsuitable for diagnostic purposes and a miss rate in the colon exceeding 30% [12]. Temporary visual interferences and tumbling movements of the CEs include oblique-forward movement, oblique-reverse movement, perpendicular and rotational movements [13]. In addition, rapid colonic motility could result in incomplete imaging considering that most of the commercial CEs are designed to acquire images at a pre-fixed frame rate, usually 2 frames per second (FPS) [14]. Moreover, tumbling movement by peristalsis also limits the visual field and causes failure to catch significant lesions or grossly distorts the perceived dimensions of polyps [15].

The PillCam Colon capsule (Given Imaging, Yoqneam, Israel) is the only CE currently in use for colonic investigation. In the most recent study of 56 patients, colon capsule endoscopy (CCE) was followed by conventional colonoscopy (CSPY). Polyp detection rate (per patient) was 50% (n=28) for CSPY and 62% (n=35) for CCE. For relevant polyps (>5 mm) there was a correspondence in the detection rates of both methods (p<0.05). The mean sensitivity was 50% (95% confidence interval [Cl], 19 to 81), the mean specificity was 76% (95% CI, 63 to 86), the positive predictive value (PPV) was 20% and the negative predictive value (NPV) was 93% [16]. These results indicate the general problem of CE tumbling during its transit in the colon and the need for CE stabilization [15]. Recent submissions on self-stabilization capsule endoscopy systems seem to overcome this issue.

Recent US patent applications address various aspects of capsule endoscopy, including expandable capsule endoscopes:
1. 20110245611 EXPANDABLE CAPSULE ENDOSCOPE AND EXPANDABLE CAPSULE ENDOSCOPY SYSTEM
2. 20110245604 CAPSULE ENDOSCOPE AND CAPSULE ENDOSCOPY SYSTEM
3. 20110137674 APPARATUS AND METHOD FOR VERIFYING PROCEDURE COMPLIANCE
4. 20110098532 METHOD FOR POSITIONING AN ENDOSCOPY CAPSULE THAT CAN BE MAGNETICALLY NAVIGATED USING A SOLENOID SYSTEM
5. 20110060189 Apparatus and Methods for Capsule Endoscopy of the Esophagus
6. 20110054255 METHOD FOR CONTROLLING THE MOVEMENT OF AN ENDOSCOPIC CAPSULE
7. 20110054254 COIL ARRANGEMENT FOR GUIDING A MAGNETIC ELEMENT IN A WORKING SPACE
8. 20110044515 DEVICE, SYSTEM AND METHOD FOR AUTOMATIC DETECTION OF CONTRACTILE ACTIVITY IN AN IMAGE FRAME
9. 20100274086 POSITION CONTROL OF MEDICAL APPLIANCES IN THE HUMAN BODY BY MEANS OF PHASE DIFFERENCE MEASUREMENT
10. 20100268025 APPARATUS AND METHODS FOR CAPSULE ENDOSCOPY OF THE ESOPHAGUS
11. 20100234685 COIL SYSTEM FOR THE CONTACT-FREE MAGNETIC NAVIGATION OF A MAGNETIC BODY IN A WORKING SPACE
12. 20100152534 CAPSULE ENDOSCOPY SYSTEM, MEDICAL SYSTEM, AND OPERATION METHOD OF MEDICAL SYSTEM
13. 20100056864 CAPSULE-TYPE IMAGE PHOTOGRAPHING APPARATUS AND ENDOSCOPY USING THE SAME
14. 20100030022 METHOD AND SYSTEM WITH ENCAPSULATED IMAGING AND THERAPY DEVICES, COUPLED WITH AN EXTRACORPOREAL IMAGING DEVICE
15. 20090187071 CAPSULE ENDOSCOPE SYSTEM AND METHOD FOR IMPLEMENTING TIME SHIFT FUNCTION THEREIN
16. 20090105541 ENDOSCOPIC CAPSULE
17. 20080255409 PROCESS TO IMPROVE THE PROFITABILITY OF TESTS OR TREATMENTS WITH ENDOSCOPY CAPSULES
18. 20080249362 Endoscope System with a Disposal Sheath
19. 20080242931 CAPSULE ENDOSCOPIC SYSTEM AND OPERATION CONTROL METHOD OF CAPSULE ENDOSCOPE
20. 20080207999 Endoscopic Capsule
21. 20080167525 Magnetically Propelled Capsule Endoscopy
22. 20070109118 Method and Apparatus for Locating and Tracking Persons
23. 20070038063 Method for determining the position and orientation of an endoscopy capsule guided through an examination object by using a navigating magnetic field generated by means of a navigation device
24. 20070021654 Magnetically navigable endoscopy capsule with a sensor for acquiring a physiological variable
25. 20070010709 Endoscopy capsule
26. 20050187479 Cable-free endoscopy method and system for determining in vivo position and orientation of an endoscopy capsule
27. 20050171418 Capsule endoscopy system
28. 20050096526 Endoscopy device comprising an endoscopy capsule or an endoscopy head with an image recording device, and imaging method for such an endoscopy device
29. 20050062562 Magnetically navigable device with associated magnet element
30. 20050043583 Endoscopy apparatus
31. 20040199054 Magnetically propelled capsule endoscopy
32. 20040174258 Method and apparatus for locating and tracking persons
33. 20040106849 Multi-functional, bi-directional communication telemetry capsule
34. 20060178557 Self-stabilizing encapsulated imaging system

2.2.6. Computed Tomographic Colonography (Virtual Colonoscopy).

It has been suggested that virtual colonoscopy performed with a computed tomography is an accurate screening method for the detection of colorectal neoplasia in asymptomatic average-risk adults and compares favorably with optical colonoscopy in terms of the detection of clinically relevant lesions. In a 2003 study Pickhardt et al. (N Engl J Med 2003; 349:2191-2200) suggested that the sensitivity of virtual colonoscopy for adenomatous polyps was 93.8 percent for polyps at least 10 mm in diameter, 93.9 percent for polyps at least 8 mm in diameter, and 88.7 percent for polyps at least 6 mm in diameter. The sensitivity of optical colonoscopy for adenomatous polyps was 87.5 percent, 91.5 percent, and 92.3 percent for the three sizes of polyps, respectively. The specificity of virtual colonoscopy for adenomatous polyps was 96.0 percent for polyps at least 10 mm in diameter, 92.2 percent for polyps at least 8 mm in diameter, and 79M percent for polyps at least 6 mm in diameter. Some of the recent US patent applications related to this method are listed below. Virtual colonoscopy has its disadvantages, in terms of patient compliance with Barium and radiation.
1. 20110122068 VIRTUAL COLONOSCOPY NAVIGATION METHODS USING A MOBILE DEVICE
2. 20110013815 METHOD AND DEVICE FOR PROVIDING A SEGMENTED VOLUME DATA RECORD FOR A VIRTUAL COLONOSCOPY, AND COMPUTER PROGRAM PRODUCT
3. 20100268154 APPARATUS AND METHOD FOR AUTOMATING AN ENEMA WITH CONTROLLED DISTENSION
4. 20080117210 VIRTUAL ENDOSCOPY
5. 20080069419 Virtual fly over of complex tubular anatomical structures
6. 20070270682 Teniae coli guided navigation and registration for virtual colonoscopy
7. 20070098633 VIRTUAL COLONOSCOPY WITH RADIOLABELED PHOSPHOLIPID ETHER ANALOGS
8. 20060013767 Virtual colonoscopy with radiolabeled phospholipid ether analogs
9. 20050107691 Methods for digital bowel subtraction and polyp detection
10. 20040167400 Method and apparatus for improving a virtual colonoscopy and A CT angiography
11. 20020097320 System for digital bowel subtraction and polyp detection and related techniques
12. 20020045153 System and method for performing a three-dimensional virtual segmentation and examination with optical texture mapping
13. 20020039400 System and method for performing a three-dimensional examination with collapse correction

2.3. Colon Cleansing.

Adequate colon cleansing is essential for reliable diagnostic and surgical colon procedures. Accuracy and safety of diagnostic testing and proper surgical procedures depend on good colon preparation. Patient compliance is enhanced by simplicity and well-tolerated methods. Several colon-cleansing methods are available (Toledo a. Dipalma, Alimentary Pharmacology & Therapeutics, 2001; 15(5), 605-611). Diet and cathartic regimens utilize clear liquids or diets designed to leave a minimal colonic residue. Laxatives, cathartics and enemas are employed. Gut lavage solutions are osmotically balanced electrolyte lavage products. Oral sodium phosphate solutions and tablets are available and are attractive because of good efficacy with a small volume of administration. Nevertheless, colonoscopy and particularly colon-targeting capsule endoscopy often suffer from inadequate colon preparation. Some of the recent US patent applications related to colon preparation are listed below.
1. 20110076339 Colon Cleansing Method and Kit
2. 20100208956 ELECTRONIC COLON CLEANSING METHOD FOR VIRTUAL COLONOSCOPY
3. 20090258090 COLON CLEANSING SOLUTION
4. 20090060942 Detoxification Composition and Method of Detoxifying the Body
5. 20080220087 COLON CLEANSING COMPOSITION AND METHOD
6. 20080145445 Aspartame and Citrate Flavored Phosphate Salt Laxative
7. 20070298100 Compressed Pharmaceutical Compositions Comprising Peg and Electrolytes
8. 20070298008 Method of bowel cleansing
9. 20070196322 Method of preparing the colon for virtual colonoscopy
10. 20060073214 Colon cleansing composition and method
11. 20060051428 Aspartame and citrate flavored phosphate salt laxative
12. 20050256464 Apparatus for washing the colon
13. 20050064043 Colon cleansing composition and method
14. 20040241134 Colon cleansing compositions and methods of use therefor
15. 20040191213 Method of bowel cleansing
16. 20030202957 Salt solution for colon cleansing
17. 20030195481 Method and apparatus for cleansing the colon
18. 20030181886 Multi-use hygienic cleansing device

2.4. Pseudobezoars.

Recently proposed pseudobezoar technology has been suggested for the treatment of obesity and for controlled drug delivery in the body (see e.g. US Patent Application Nos. 20100215732, 20100145316, 20090035367). In the present application we suggest to utilize these retaining devices as platforms for (a) colonic cleansing performed by the sponge-like cleansing action performed by a colon-targeted pseudobezoar in preparation for subsequent colonic biopsy or colonoscopy, or as an independent procedure; and (b) colon biopsy performed from the inside of the colon by a colon-targeted pseudobezoar which will be in contact with the colonic walls in a friction-like fashion severe enough to collect tissue samples, but moderate enough not to cause excessive or abnormal bleeding or mucosal damage. This “artificial stool” will enable: (a) improved colon cleansing; and (b) generalized biopsy from the entire organ (without actually having the information from which exact location in the organ the tissue samples have been collected).

2.5. Voids in Technology.

The closest patent application to our method is US Patent Application 20050266074. It discloses a colon-targeting ingestible device platform designed to recognize its entry to the colon and expand in the colon, ultimately aiming at improved imaging of the colon walls. On approaching the external anal sphincter muscle, the ingestible pill may contract or deform, for elimination. Colon recognition may be based on a structural image, based on the differences in diameters between the small intestine and the colon, and particularly, based on the semilunar fold structure, which is unique to the colon. Additionally or alternatively, colon recognition may be based on a functional image, based on the generally inflammatory state of the vermiform appendix. Additionally or alternatively, pH, flora, enzymes and (or) chemical analyses may be used to recognize the colon. The imaging of the colon walls may be functional, by nuclear-radiation imaging of radionuclide-labeled antibodies, or by optical-fluorescence-spectroscopy imaging of fluorescence-labeled antibodies. Additionally or alternatively, it may be structural, for example, by visual, ultrasound or MRI means. Due to the proximity to the colon walls, the imaging is claimed to be advantageous to colonoscopy or virtual colonoscopy, as it is designed to distinguish malignant from benign tumors and detect tumors even at their incipient stage. Various sensors and detectors are envisioned to be embedded within the expandable colonic structure, including e.g. radioactive-emission detectors, fluorescence detectors, ultrasound detectors, MRI detectors, still and video cameras operating in the visible and/or infrared light ranges, temperature detectors, and impedance detectors.

Our technology also offers a colon-targeting expandable structure, but it has 5 distinct features:
1. Its expansion is facilitated by a permeable, mesh-like gauze structure which is in constant contact with the walls of the colon;
2. The expansion is provided by swellable granules of an appropriate biocompatible polymer (e.g. polyacrylic acid) which swell individually but do not fuse into each other, thus not forming a uniform mass non-permeable to gases and liquids causing colonic obstruction;
3. The design of the entire device is such that the mesh-like gauze structure can exert relatively constant pressure on the colonic walls from the moment it reaches its final dimensions, until it exits the organ, thus providing either gentle contact (for maximal absorption of colonic fluids in a colon cleansing setup) or abrasive contact (for scraping maximally well the colonic walls while retaining the scrapings within the structure, without, however, damaging the colonic walls);
4. Our technology aims at collecting samples of tissue and bodily fluids, to be expelled from the body and analyzed later, rather than detecting colon pathologies in situ via detectors as disclosed in the cited US Patent Application 20050266074;
5. There is an embedded mechanism to disintegrate the entire structure, either spontaneously or on demand. The disintegration can be controlled chemically, mechanically, electronically, or by any other means, and prevents possible obstruction that the structure might create in the investigated organ.

2.6. Aim of Disclosure.

The aim of this disclosure is to offer a technology of creating a controllable, organ-targeting gastrointestinal pseudobezoar with the purpose (a) to cleanse the targeted gastrointestinal organ by absorbing unwanted fluids and debris from within the organ; and (b) to scrape the organ from inside in order to collect maximal diagnostic information for further processing.

### SUMMARY OF THE INVENTION

According to a first broad aspect of this invention, there is provided an orally administrable implement for expanding in a gastrointestinal organ of an animal, including a mammal, to fill a space in the organ, the implement including:
a fluid-permeable expandable container having a first dimension and a second dimension packaged in an organ-targeting shell cover;
at least one molecule cluster comprising a swellable material contained within the container and capable of swelling when contacted with a fluid; and
a control mechanism to disintegrate the fluid-permeable expandable container on demand, when needed, or at a pre-determined moment in time.
   whereby when the implement is ingested and it reaches the targeted GI organ, the organ-targeting shell cover rapidly disintegrates, fluids from the targeted GI organ enter the fluid-permeable mesh-like container causing the molecule clusters therein to swell and the container to expand from the first dimension to the second dimension forming an intraluminal pseudobezoar, which moves inside the targeted organ as a result of natural peristalsis. In the process of its movement within the targeted gastrointestinal organ, the swollen pseudobezoar either cleanses the said organ, or scrapes (in a non-harmful way) its internal walls to collect occult blood and/or tissue samples containing DNA signatures, or both.

Preferably, the implement can be self-administrable (in the case of humans) or administrable autonomously or unaided, meaning the implement is administrable in a non-invasive fashion, without the need of any external positioning or manipulating device functionally attached to it, such as an endoscope.

Preferably, when the container has the first dimension, the implement can be retained in a capsule capable of being easily swallowed or administered autonomously. Once the capsule has dissolved and the container is released in the colon, the colonic fluids will enter the fluid-permeable, mesh-like, expandable container. When the fluid contacts the at least one swellable molecule cluster, the cluster will swell and the container will expand to the second dimension. When the container has expanded to the second dimension, it is sufficiently large so as to touch the colonic walls. The number of swellable molecule clusters in the container, their individual diameter, and their liquid-retaining and absorbing properties under various pressures, as well as the design of the container itself are made such that the swollen implement has an appropriate compliance to remain in constant touch with the colonic walls regardless of the lumen of the organ. For example, in a section of the colon where the lumen is large, the implement expands in a spherical shape to touch the walls of the organ. When the lumen of the colon is reduced, the implement elongates itself longitudinally in the organ, but it remains in contact with the colonic walls.

The organ-targeting capsule can be any gelatin capsule known in the art, for example, a DB AAA capsule made from Capsugel™, Greenwood, S.C., covered by a colon-targeting combination of Eudragit® L100-55 and Eudragit® S100 as discussed by Khan at al., Journal of Controlled Release; Volume 58, Issue 2, 29 Mar. 1999, Pages 215-222.

In one embodiment, the container is biodegradable over time. Thus, when the implement is in the colon, as a safety layer preventing colonic obstruction, the colonic fluids will cause the container to long-term biodegrade, thereby releasing the swelled clusters from the container and into the colon. In a preferred embodiment, the clusters swell to a size that does not exceed 1 cm. Preferably, the clusters swell to a size not exceeding about 0.5 cm to about 0.6 cm. In one embodiment, the clusters cannot fuse into each other either when dry or when swelled so that chances of colonic obstruction or constipation are minimized. In another embodiment, the clusters can be pre-fused when dry, to form a homogeneous structure when they swell. However, the said structure remains porous and fluid- and gas-permeable, and should be able to be taken apart by colonic peristaltic forces after the container biodegrades.

In one embodiment, the container is made of specific biodegradable woven, knitted, braided or monofilament mesh material, such as Vicryl™ (Ethicon), Monosyn™ (B Braun), polylactic acid (Ahlstrom, Helsinki, Finland), PDS II™ (Ethicon, Cornelia, Ga.) and the like, which allows fluid to permeate while having a mesh-like abrasive surface in order to scrap the colonic mucosal wall as much as possible while traversing the organ, without actually harming it. In another embodiment, the container is made from a biodegradable fluid-permeable stretchable material such as interlaced regenerated oxidized cellulose (for example, Curacel™ by CuraMedical BV, Amsterdam, Holland), or circularly knitted PDS II or/and Vicryl threads, which expands or stretches from the first dimension to the second dimension when the clusters swell, thus exerting constant and known pressure on the colonic wall.

In another embodiment, the container comprises a plurality of smaller sections, whereby each section is attached to one another by biodegradable fibers to form the container. The biodegradable fibers can be made of an absorbable biocompatible material, which can include, but is not limited to, polycaprolactone, polyglycolide, polylactide, or combinations thereof (commercially available under the names Selecture PLL™ and Selecture VEH™ by Schering-Plough Animal Health Corporation). The biodegradable fibers can further be made, for example, from any absorbable suture known in the art such as Vicryl™, Monosyn™, catgut, PDS II™ (Ethicon, Cornelia, Ga.), or any other appropriate braided or monofilament absorbable suture. Soft monofilament material or material such as regenerated oxidized cellulose (for example, Curacel) or catgut could be utilized also to avoid possible mucosal injuries.

In another embodiment, the container is made from permeable biodegradable mesh such as Vicryl™ Knitted Mesh by Ethicon, Curacel™ by CuraMedical, or Safil™ Mesh by B Braun and the mesh has radial fibers made, for example, from absorbable surgical suture such as Vicryl™, PDS II™ (Ethicon), catgut, regenerated cellulose or Monosyn™ (B Braun) woven therethrough. The radial fibers are biodegradable, hence when the fibers begin to disintegrate the volume of the container collapses, the container loses its integrity due to the gastric peristaltic forces, and the clusters are released.

In another embodiment, the pseudobezoar which has left the colon can be mechanically collected by the patient from the toilet bowl after visual recognition. However, other means of recognizing the presence of the expelled pseudobezoar in the toilet bowl can be implemented. For example, a miniature passive Radio Frequency Identification (RFID) tag can be included in the pseudobezoar, and a receiver attached to the toilet bowl (for example, in a toilet bowl sanitizer box), or mounted at another washroom location in sufficient proximity to the toilet bowl, could beep or light up if the pseudobezoar is detected in the toilet bowl. The patient then could mechanically collect the expelled pseudobezoar. Other means of automatic pseudobezoar identification are also possible, including, but not limited to, an appropriate biocompatible dye or chemical presenting a visually contrasting colour detectable in the toilet boil, magnetic-based detection solutions, sound-based recognition solutions, etc.

In yet another embodiment, the container has miniature clubs on its surface created during the manufacturing process so that when the container swells these clubs can scrub the mucosa of the colonic walls very efficiently while retaining maximal amount of tissue samples, occult blood, or both within the pseudobezoar structure as it traverses the colon.

In another embodiment, the surface of the container has a plurality of miniature brushes made of the same material as the container, the length of which can be controlled. Longer brushes can be utilized in a colon-cleansing application, while shorter brushes can be more abrasive and be applicable for colon-scraping purposes.

In one embodiment, the molecule clusters comprise a swellable material selected from the group consisting of a swelling alginates, Konjak-glucomannan, bentonite, microcrystalline hydrogels, polyolefins and various mixtures thereof. Other swellable materials that could be used include, by are not limited to, other natural clays, polyvinyl alcohol, poly(ethyloxazoline), polyvinylacetate-polyvinylalcohol copolymers, poly(2-hydroxyethylacrylate), poly(2-hydroxyethylmethacrylate), polyacrylic acid, and copolymers thereof, polysaccharides, water soluble proteins, polynucleic acids, or a combination thereof. Furthermore, if desired, the clusters comprise a swellable material that is also biodegradable, thereby further facilitating each clusters passage through the intestines. It is understood that a variety of other biocompatible super-absorbent polymers known in the art can be used to form the clusters of the present invention, for example, polymers of poly(2-hydroxyethyl methacrylate) by Aldrich, Milwaukee, Wis., or of polyacrylamide, or of an appropriately cross-linked poly(acrylic acid) (for example, one produced by Wako Pure Chemical Industries, Japan) which expand adequately in higher pH environment (5-7), but not in low pH environment (below 5).

In one embodiment, the entire pseudobezoar structure can be made disintegratable in a given period of time spent in the targeted GI organ (e.g. the colon) by the chemical degradation of the permeable container, the way it is held or sutured together, or by combination thereof. Upon disintegration the remnants of the entire structure exit the body in a natural way, through gastrointestinal peristalsis.

In another embodiment the pseudobezoar disintegration, and therefore, the moment the entire structure will start leaving the targeted organ and the body can be controlled through a control system embedded within the pseudobezoar, either in a pre-programmed fashioned, or wirelessly from outside the body. For example, a miniature microheater of the type developed by Yeom et al (The design, fabrication and characterization of a silicon microheater for an integrated MEMS gas preconcentrator, J. Micromech. Microeng., 18:12 pp, 2008) can be controlled by a wireless receiver obtaining disintegration commands from the user, or from medical professional. The obtained controlling signal from the outside world turns on the embedded microheater to melt a biocompatible surgical suture holding the pseudobezoar structure together.

Pre-programmed or user-programmable disintegration-timing options for activating the heater could include use of a pre-programmed timer with a predetermined count-down value that is triggered to start just prior to ingestion, or a programmable timer where a user can enter a particular count-down time or select from existing count-down options just prior to ingestion. Other options could employ a programmable timer where the user enters a particular point in time at which the heater is to be activated, based on an approximation of when the implement is expected to reach the target organ, as estimated just prior to ingestion.

According to a second broad aspect of the invention, there is provided an orally administrable implement for expanding in a targeted gastrointestinal organ of an animal, including a mammal, to touch the walls of the organ, comprising:
(a) an organ-targeting capsule shell arranged to rapidly disintegrate in the targeted gastrointestinal organ;
(b) a fluid-permeable expandable container contained within the organ-targeting capsule shell and having a first dimension, the fluid-permeable expandable container being expandable to a second dimension of sufficient size to contact organ walls of the targeted gastrointestinal organ from inside said targeted gastrointestinal organ;
(c) at least one cluster contained within the container comprising a swellable material arranged to swell when contacted with a fluid;
   wherein the implement is ingestible for subsequent disintegration of the organ-targeting capsule shell inside the targeted gastrointestinal organ, whereupon the fluid in the said organ enters the fluid-permeable expandable container causing the at least one cluster therein to swell and the container to expand from the first dimension to the second dimension so that the swollen implement touches the walls of the targeted gastrointestinal organ.

According to a third broad aspect of the invention, there is provided an orally administrable implement for expanding in a targeted gastrointestinal organ of an animal, including a mammal, to touch the walls of the organ, comprising:
(a) an organ-targeting capsule shell arranged to rapidly disintegrate in the targeted gastrointestinal organ;
(b) a fluid-permeable expandable container contained within the organ-targeting capsule shell and having a first dimension, the fluid-permeable expandable container being expandable to a second dimension of sufficient size to contact organ walls of the targeted gastrointestinal organ from inside said targeted gastrointestinal organ;
(c) at least one cluster contained within the container and each comprising a swellable material contained within the container and arranged to swell when contacted with a fluid;
(d) an embedded control system that controls a device arranged to cause disintegration of the fluid permeable expandable container.

According to a fourth broad aspect of the invention, there is provided a method of obtaining samples from walls of a targeted gastrointestinal organ of an animal, including a mammal, the method comprising:
(a) orally administering an implement comprising a capsule shell, a fluid-permeable expandable container contained within the organ-targeting capsule shell and having a first dimension inside said organ-targeting capsule shell, and at least on cluster contained within the container and each comprising a swellable material arranged to swell when contacted with a fluid;
(b) allowing the implement to reach targeted gastrointestinal organ, whereupon the capsule shell rapidly disintegrates and fluid in the said organ enters the fluid-permeable expandable container and causes the at least one cluster therein to swell and the container to expand from the first dimension to a second dimension in which the fluid-permeable expandable container contacts the walls of the targeted gastrointestinal organ, thereby collecting sample material from said walls on the fluid-permeable expandable container;
(c) causing or allowing the fluid-permeable expandable container to disintegrate into pieces, thereby releasing the at least one cluster from the fluid-permeable expandable container; and
(d) allowing the pieces of the fluid-permeable expandable container to pass through and exit the gastrointestinal system;
(e) collecting at least one of the pieces of the fluid-permeable expandable container, and the sample material collected thereon.

According to a fifth broad aspect of the invention, there is provided a method of cleansing walls of a targeted gastrointestinal organ of an animal, including a mammal, the method comprising:
(a) orally administering an implement comprising a capsule shell, a fluid-permeable expandable container contained within the organ-targeting capsule shell and having a first dimension inside said organ-targeting capsule shell, and at least one cluster contained within the container and each comprising a swellable material arranged to swell when contacted with a fluid;
(b) allowing the implement to reach targeted gastrointestinal organ, whereupon the capsule shell rapidly disintegrates and fluid in the said organ enters the fluid-permeable expandable container and causes the at least one cluster therein to swell and the container to expand from the first dimension to a second dimension in which the fluid-permeable expandable container contacts the walls of the targeted gastrointestinal organ, thereby collecting material from said walls on the fluid-permeable expandable container;
(c) causing or allowing the fluid-permeable expandable container to disintegrate, thereby releasing the at least one cluster from the fluid-permeable expandable container into the gastrointestinal system.

A sixth broad aspect of the invention extends to use of an ingestible implement to cleanse a targeted gastrointestinal organ of an animal, including a mammal, wherein the implement comprises:
(a) an organ-targeting capsule shell arranged to rapidly disintegrate in the targeted gastrointestinal organ;
(b) a fluid-permeable expandable container contained within the organ-targeting capsule shell and having a first dimension, the fluid-permeable expandable container being expandable to a second dimension of sufficient size to contact organ walls of the targeted gastrointestinal organ from inside said targeted gastrointestinal organ;
(c) at least one cluster contained within the container and each comprising a swellable material arranged to swell when contacted with a fluid.

A seventh broad aspect of the invention extends to use of an ingestible implement to obtain samples from walls of a targeted gastrointestinal organ of an animal, including a mammal, wherein the implement comprises:
(a) an organ-targeting capsule shell arranged to rapidly disintegrate in the targeted gastrointestinal organ;
(b) a fluid-permeable expandable container contained within the organ-targeting capsule shell and having a first dimension, the fluid-permeable expandable container being expandable to a second dimension of sufficient size to contact organ walls of the targeted gastrointestinal organ from inside said targeted gastrointestinal organ;
(c) at least on cluster contained within the container and each comprising a swellable material arranged to swell when contacted with a fluid.

For purposes of summarizing the invention and the advantages achieved over the prior art, certain objects and advantages of the invention have been described above. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The present invention, both as to its organization and manner of operation, may best be understood by reference to the following description, and the accompanying drawings of various embodiments wherein like reference numerals are used throughout the several views, and in which:

FIG. 1A is a schematic view of one embodiment of an orally administrable implement according to the invention, where the container is in the first dimension and swellable clusters are unswelled. The entire implement is encapsulated within a gelatin capsule covered with a colon-targeting Eudragit combination.

FIG. 1B is a schematic view of the orally administrable implement of FIG. 1A in the expanded second dimension as a result of swellable clusters swelling, to form a pseudobezoar. The colon-targeting capsule has already disintegrated.

FIG. 1C is a schematic view of the orally administrable implement of FIG. 1A in the expanded second dimension as a result of the swellable clusters swelling, to form a pseudobezoar. The colon-targeting capsule has already disintegrated. The container holding the swellable clusters together has started disintegrating due to chemical ageing, and the entire implement has fallen apart, with the swollen clusters becoming loose in the gastrointestinal tract.

FIG. 2A is a schematic view of one embodiment of an orally administrable implement according to the invention, where the container is in the first dimension and swellable clusters are unswelled. Within the container resides a carrier, carrying a control system and a controllable microheater, and a thread threaded through the microheater holding the entire implement together. The implement is encapsulated within a colon-targeting gelatin capsule covered by an Eudragit combination.

FIG. 2B is a schematic view of the orally administrable implement of FIG. 2A in the expanded second dimension as a result of at least one swellable cluster swelling, to form a pseudobezoar. The carrier carrying the control system, the controllable microheater, and the thread threaded through the microheater holding the entire implement together is embedded within this pseudobezoar.

FIG. 2C is a schematic view of the orally administrable implement of FIG. 2A in the expanded second dimension as a result of the swellable clusters swelling, to form a pseudobezoar. The carrier is embedded within this pseudobezoar. The container holding swellable clusters together has started disintegrating due to the control system actuating the microheater, which in turn has melted the thread holding the pseudobezoar container together. Thus, the entire implement has fallen apart, with the swollen clusters and the carrier becoming loose in the gastrointestinal tract.

FIG. 3A represents a possible design of the surface structure of the permeable container, having mesh-like surface made of crossing-over threads for improved scrubbing of the colonic walls.

FIG. 3B represents a possible design of the surface structure of the permeable container, having clubs for improved scrubbing of the colonic walls.

FIG. 3C shows a possible design of the surface structure of the permeable container, having short brushes for improved scrubbing of the colonic walls.

FIG. 3D shows a possible design of the surface structure of the permeable container, having long brushes for improved cleansing of the colonic walls.

FIG. 4 shows a block-diagram of the remotely-controlled microheater-based disintegration mechanism.

### DETAILED DESCRIPTION OF THE DRAWINGS

One embodiment of an orally administrable implement for expanding in a stomach or other targeted gastrointestinal organ of an animal, including mammal, to swell in the targeted gastrointestinal organ for the purpose of cleansing it, or to scrub it in order to collect occult blood, tissue samples, or both, includes a fluid-permeable expandable carrier/container having a first collapsed, folded or otherwise compact dimension and a second dimension achievable by subsequent expansion, and a plurality of clusters comprising a swellable material contained within the container and capable of swelling when contacted with a fluid. In addition, a control system may be embedded within the expandable container along with its periphery, for example including its power supply, Radio Frequency transceiver, and electronically-controllable microheater. When at least one swellable cluster contact the fluid in the targeted organ, the at least one swellable cluster swells and the container expands from the first dimension, which is generally of a size that allows the implement to fit in an organ-targeting capsule, to the second dimension, which is generally of a size and compliance that touches the walls of the targeted organ with a relatively constant contact force.

After the desired and controllable amount of time has passed, and in cases of possible obstruction, the integrity of the container is compromised in a timed fashion, for example, by the disintegration of the container by degradation of the container walls themselves of threads or fibres holding distinct pieces of the container walls together, and the swelled clusters are released from the container. This disintegration can allow the disintegrated parts of the container, the control system (which is appropriately shelled in a biocompatible shell) and the swelled clusters, to now pass through the pylorus, and empty from the stomach. Preferably, each expanded or swelled cluster and the control system shell do not exceed 1 cm in diameter. When the said clusters and the control system shell are released from the container, they can individually pass through the gastrointestinal system and exit it in a harmless fashion.

The form of container can vary widely and disintegration of the container can be due to the container comprising a biodegradable material or comprising a plurality of sections held together by biodegradable materials such as fibers, absorbable surgical sutures or absorbable gauze. The actual timing of the disintegration of the container can be estimated by knowing the reduction in the tensile strength of the biodegradable fibers or gauze used to hold the sections of the container together after ingestion. That is, the length of time for which the container will remain intact can be controlled through selection from among biodegradable container materials of different known degradation times (i.e. time of stability before degradation to a point where the container breaks apart).

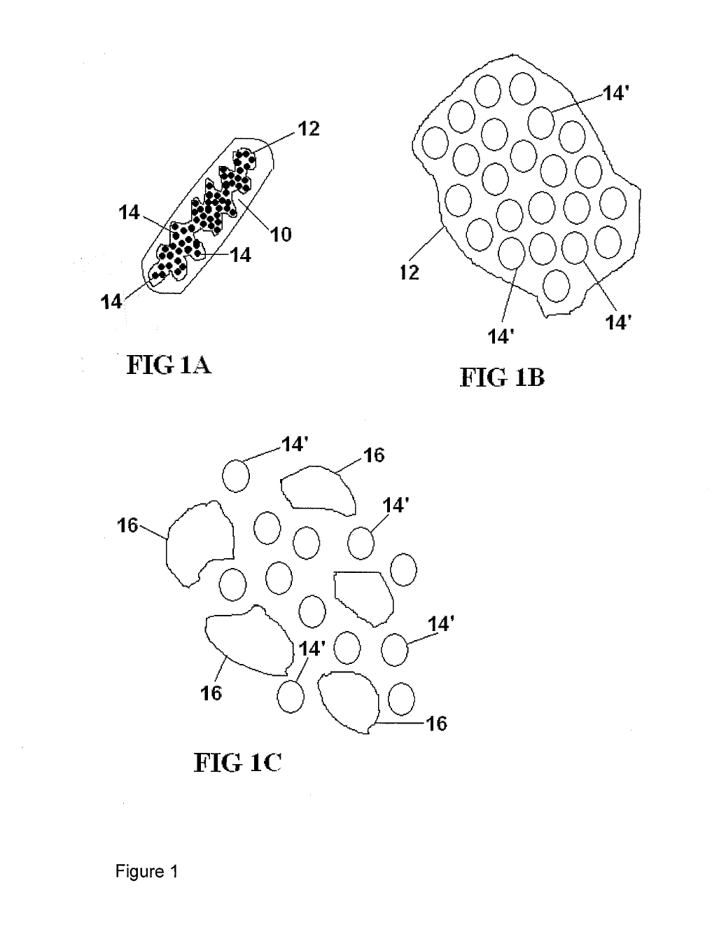

In one embodiment of the present invention, illustrated in FIGS. 1A, 1B and 1C, the orally administrable implement, referred to generally as 10, comprises a container 12, shown here in a folded, compact, first dimension. In this embodiment, container 12 is made from a biodegradable material that allows for the passage of fluid into its interior 13, for example, a permeable biodegradable mesh such as Vicryl™ Knitted Mesh by Ethicon, Curacel™ by CuraMedical, or Safil™ Mesh by B Braun. Further contained in the interior 13 of container 12 is at least one cluster 14 comprising a swellable material, whereby each swellable cluster is capable of swelling when contacted with fluid such as gastric fluid found in the stomach. For example, clusters 14 can comprise Aquagel™ by Akina Inc., West Lafayette, Ind., polyacrylate, or PGX granules (Natural Factors, Vancouver, BC, Canada). In FIG. 1A, swellable clusters 14 are shown prior to contact with fluid, i.e., in their non-swelled form.

FIG. 1B shows implement 10 of FIG. 1A in its expanded form, after it has been delivered into the stomach and gastric fluid has been allowed to contact it. Container 12 is now shown in its second, expanded dimension, such that the implement 10 can no longer exit the stomach through the pylorus. The swellable clusters 14′ are now shown in their swelled state due to the gastric fluid seeping through the container 12. The swelling of clusters 14′ then causes container 12 to expand to the second dimension. Preferably, the swelled clusters 14′ become spherical bodies not exceeding about 1 cm in diameter. The swellable clusters can be made of various substances, for example, appropriately cross-linked poly(acrylic acid) or poly(2-hydroxyethyl methacrylate). Preferably, they are of size not permitting their exit from the container when dry or maximally expanded, and preferably not exceeding about 1 cm when swollen in gastrointestinal fluid. In addition, preferably, they cannot grow any bigger in the intestines to prevent them causing obstruction.

FIG. 1C represents the released pieces 16 of the container 12 in FIG. 1B once the container biodegrades, each piece 16 of which is of size precluding the possibility of creating obstruction in the targeted GI organ. The container pieces 16 along with the swelled clusters 14′ are released in the stomach, so that they can be propelled out of the body by natural peristalsis in a harmless fashion.

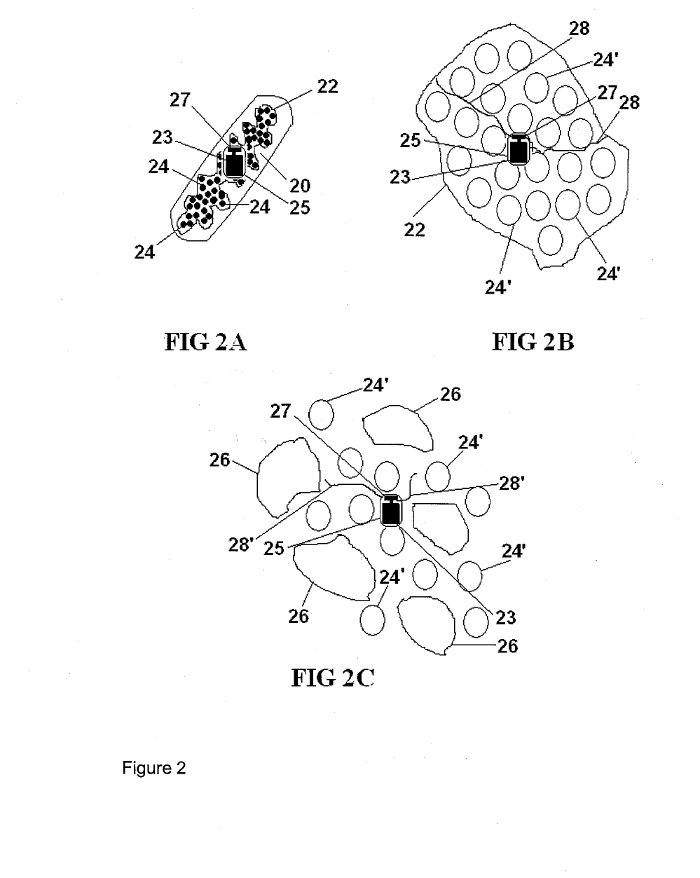

FIG. 2 shows a similar build-up of at least one pseudobezoar cluster, 24, from condensed version in FIG. 2A to swollen version, 24′, in FIG. 2B. A control system, 25, is embedded within the at least one cluster, and resides in the pseudobezoar based platform in the gauze, 22, encapsulated in a biocompatible shell 23 together with a controllable microheater 27. The control system in this embodiment controls the microheater 27 that enables the disintegration of the platform/implement which is held together by the internal threads or sutures 28 that pass through the controllable microheater 27. The thread is stitched through multiple pieces that are interconnected by this threading to form the overall container, and its travel also passes through the heater. The thread need not necessarily pass through the clusters of swellable material, as they are secured within the container by the closure of the container walls around them until the time of container disintegration. As outlined in FIG. 2C, the thread 28′, which holds the container and its contents, the swollen polymer clusters 24′ and the control system 25 is severed by the microheater 27 under the control of the microheater 25. Once the suture is not intact, the swollen polymer clusters 24′, the control system 25 and the now dysfunctional microheater 27 exit the body through the GI tract in their biocompatible shell 23, just like food chime or stool would.

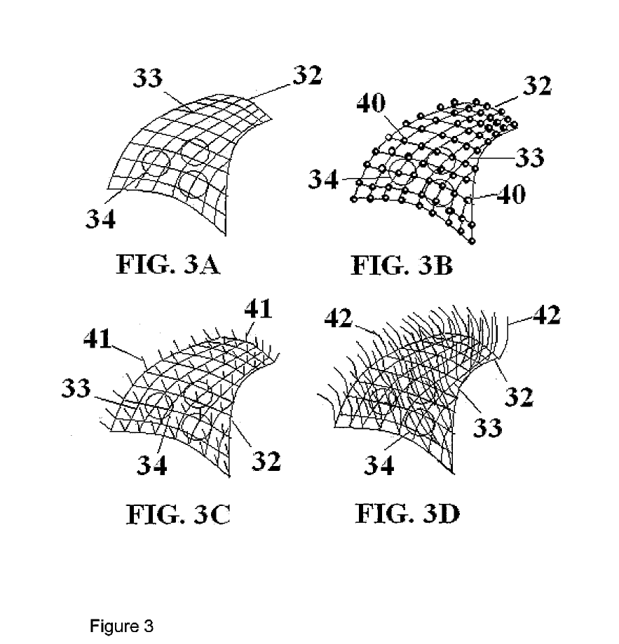

FIG. 3 shows different implementations of the surface area of the container. FIG. 3A shows the container 32 implemented with meshed crossing-over threads 33. The swollen clusters 34 inside the container 32 are of size that they cannot exit the mesh-like structure of the container. FIG. 3B shows also the container 32 implemented with meshed crossing-over threads 33, but at each cross-over there is a formed a club 40 protruding outward from the mesh wall of the container, for example in the form of a knot or tie secured to the crossing threads of the mesh at the intersection thereof, or a member tied to this intersection point. The swollen clusters 34 inside the container 32 are of size that they cannot exit the mesh-like structure of the container. FIG. 3C shows the container 32 implemented with meshed crossing-over threads 33, but at each cross-over there is a short out-of-plane thread 41 attached to extend outward from the mesh walls of the container. The plurality of these short out-of-plane threads increase the scrubbing capabilities of the surface of the container 32 by providing more contact area at the container exterior for contact with the walls of the target organ. The swollen clusters 34 inside the container 32 are of size that they cannot exit the mesh-like structure of the container. FIG. 3D shows the container 32 implemented with meshed crossing-over threads 33, but at each cross-over there is a longer out-of-plane thread 42 attached. The plurality of these longer out-of-plane threads further increase the cleansing capabilities of the surface of the container 32. The swollen clusters 34 inside the container 32 are of size that they cannot exit the mesh-like structure of the container.

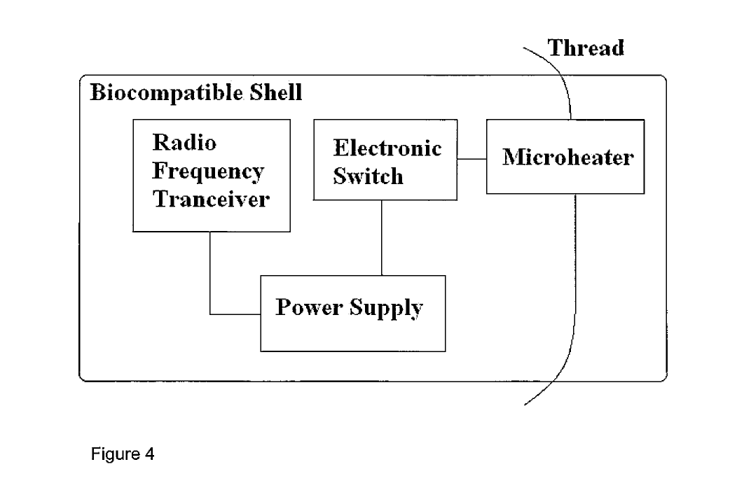

FIG. 4 shows a block-diagram of one possible implementation of a remotely-controlled system for disintegrating the entire pseudobezoar implement in case of possible intestinal obstruction. A radio-frequency transceiver is remotely controlled from outside the body so that a miniature microheater is power-supplied via an electronic switch. The microheater then melts a thread that holds the entire permeable container together. This control system is encapsulated in a biocompatible shell and is positioned inside the permeable container (see also FIG. 2).

Embodiments that include a micro-heater or other device for user-controlled start of the disintegration process provide the advantage of being able to trigger early disintegration in the event that the implement becomes lodged and creates a blockage before reaching the intended expansion site at the target organ. Embodiments with such triggered disintegration may rely on this remote controlled option for disintegration of the container in the target organ after expansion, or may rely on non-triggered, automatic degradation of one or more biodegradable materials of the container itself.

When the implement is used for the purpose of cleansing the target organ through the contact of the expanded container with the organ walls, the container itself may be made wholly of biodegradable with a suitable degradation time to keep the container intact long enough to accommodate arrival at the organ, and expansion therein against the organ walls. After such time, the container will degrade to the point of disintegration without outside intervention, allowing the swollen clusters released by the container disintegration, and any container remnants, to continue on through the gastrointestinal system for natural passage and unaided exit from the body.

Alternatively, the cleansing implement may use a container made up of distinct pieces of material interconnected by biodegradable thread that has a suitable degradation time to keep the pieces of the container intact with one another long enough to accommodate arrival at the organ, and expansion therein against the organ walls. After such time, the threads will degrade to the point of disintegrating the container through release of the pieces from one another, without requiring the use of a heater or other device to initial the disintegration process. The threaded-together container pieces may also be made of biodegradable material, or if not, at least be sufficiently small for safe passage through the gastrointestinal tract without obstruction or harm to same. Even where the pieces are biodegradable, the pieces are preferably of such unhazardous size. Where the pieces are biodegradable, the material may be selected to have a longer degradation time than the thread to ensure the container remains intact until the thread itself has sufficiently degraded to destroy or sufficiently weaken the connections between the pieces.

A further alternative of the cleansing implement again uses a container formed of threaded together pieces and an embedded micro-heater to melt away the thread, in which case the thread itself may be biodegradable or not. Again, the pieces themselves may be biodegradable, or not.

When the implement and its contact with the organ walls is instead used for the purpose of sample collection, as opposed to mere cleansing or scraping alone, pieces of the container accordingly need to remain intact throughout the full travel through the gastrointestinal system so that the material collected on the container from the organ walls can be retrieved upon successful passage of the disintegrated implement form the body. Accordingly, non-biodegradable container pieces of sufficiently small size, or biodegradable pieces of long enough degradation time are used for sample-collecting versions of the implement.

1. An orally administrable implement for expanding in a targeted gastrointestinal organ of an animal, including a mammal, to touch the walls of the organ, comprising:
(a) an organ-targeting capsule shell arranged to rapidly disintegrate in the targeted gastrointestinal organ;
(b) a fluid-permeable expandable container contained within the organ-targeting capsule shell and having a first dimension, the fluid-permeable expandable container being expandable to a second dimension of sufficient size to contact organ walls of the targeted gastrointestinal organ from inside said targeted gastrointestinal organ;
(c) at least one cluster contained within the container comprising a swellable material arranged to swell when contacted with a fluid;
wherein the implement is ingestible for subsequent disintegration of the organ-targeting capsule shell inside the targeted gastrointestinal organ, whereupon the fluid in the said organ enters the fluid-permeable expandable container causing the at least one cluster therein to swell and the container to expand from the first dimension to the second dimension so that the swollen implement touches the walls of the targeted gastrointestinal organ.

2. The implement of claim 1 having attachments on the surface of the fluid-permeable expandable container in order to increase the contact surface area between the walls of the targeted gastrointestinal organ and the surface of the said container.

3. The implement of claim 1 comprising an embedded control system that controls a device arranged to cause disintegration of the fluid-permeable expandable container.

4. The implement of claim 3 in which the control system is pre-programmed to cause the disintegration of the entire implement at a predetermined time.

5. The implement of claim 3 in which the control system is pre-programmed to cause the disintegration of the fluid permeable expandable container after a predetermined amount of time.

6. The implement of claim 1 in which the control system controls a microheater to melt a material holding the fluid permeable expandable container together.

7. The implement of claim 6 in which the control system and the microheater, are encapsulated in a biocompatible shell embedded within the fluid-permeable container.

8. The implement of claim 1 wherein the fluid permeable expandable container is formed wholly of biodegradable material.

9. The implement of claim 1 wherein the fluid permeable expandable container comprises sections made up of a first material and held together by a different second material that is biodegradable.

10. The implement of claim 1 comprising an embedded identification mechanism configured to provide a signal upon expulsion from the colon for the purpose of recognizing that said expulsion has occurred in order to enable collection and further processing.

11. The implement of claim 10 wherein the embedded identification mechanism comprises a radio-frequency identification unit configured to communicate with a receiver mounted in a washroom location.

12. The implement of claim 10 wherein the embedded identification mechanism comprises a coloring agent for detection of said expulsion by visual recognition of a color displayed by said coloring agent.

13-15. (canceled)

16. The implement of claim 3 in which the control system is remotely and wirelessly controlled to cause the disintegration of the fluid permeable expandable container at a desired moment in time.

17-19. (canceled)

20. A method of cleansing, or obtaining samples from, walls of a targeted gastrointestinal organ of an animal, including a mammal, the method comprising:
(a) orally administering an implement comprising an organ-targeting capsule shell, a fluid-permeable expandable container contained within the organ-targeting capsule shell and having a first dimension inside said organ-targeting capsule shell, and at least one cluster contained within the container comprising a swellable material arranged to swell when contacted with a fluid;
(b) allowing the implement to reach targeted gastrointestinal organ, whereupon the capsule shell rapidly disintegrates and fluid in the said organ enters the fluid-permeable expandable container and causes the at least one cluster therein to swell and the container to expand from the first dimension to a second dimension in which the fluid-permeable expandable container contacts the walls of the targeted gastrointestinal organ, thereby collecting sample material from said walls on the fluid-permeable expandable container;
(c) causing or allowing the fluid-permeable expandable container to disintegrate into pieces, thereby releasing the at least one cluster from the fluid-permeable expandable container.

21. The method of claim 27 wherein step (e) comprises first recognizing a signal generated in response to expulsion of the pieces of the fluid permeable container from the colon, and then collecting said at least one of the pieces in response to recognition of said signal

22. The method of claim 21 wherein, in step (e), the signal is generated in response to automatic detection of an RFID unit that was embedded in the implement prior to ingestion thereof.

23. The method of claim 21 wherein, in step (e), the signal comprises display of a color by a coloring agent added to the implement prior to ingestion thereof.

24. (canceled)

25. Use of an ingestible implement to cleanse, or obtain samples from, walls of a targeted gastrointestinal organ of an animal, including a mammal, wherein the implement comprises:
(a) an organ-targeting capsule shell arranged to rapidly disintegrate in the targeted gastrointestinal organ;
(b) a fluid-permeable expandable container contained within the organ-targeting capsule shell and having a first dimension, the fluid-permeable expandable container being expandable to a second dimension of sufficient size to contact organ walls of the targeted gastrointestinal organ from inside said targeted gastrointestinal organ;
(c) at least one cluster contained within the container comprising a swellable material arranged to swell when contacted with a fluid.

26. (canceled)

27. The method of claim 20 further comprising:
(d) allowing the pieces of the fluid-permeable expandable container to pass through and exit the gastrointestinal system; and
(e) collecting at least one of the pieces of the fluid-permeable expandable container, and the sample material collected thereon.
